(19) United States
(12) Patent Application Publication
(10) Pub. No.: US 2026/0013801 A1
(43) Pub. Date: Jan. 15, 2026

(54) COMPUTER-IMPLEMENTED METHODS FOR PREDICTING GLUCOSE VALUES, DATA PROCESSING SYSTEM, MEDICAL SERVER, AND USER DEVICE

(71) Applicants: Roche Diabetes Care, Inc., Indianapolis, IN (US); International Business Machines Corporation, Armonk, NY (US)

(72) Inventors: Paul J. Galley, Cumberland, IN (US); Christian Ringemann, Mannheim (DE); Yannick Klopfenstein, Neuchatel (CH); Patrick Lustenberger, Herrliberg (CH); Ajandek Peak, München (DE); Eemeli Leppäaho, Espoo (FI)

(21) Appl. No.: 19/337,109

(22) Filed: Sep. 23, 2025

Related U.S. Application Data
(63) Continuation of application No. PCT/EP2024/058059, filed on Mar. 26, 2024.
(60) Provisional application No. 63/493,336, filed on Mar. 31, 2023.

(30) Foreign Application Priority Data
Apr. 14, 2023 (EP) 23167970.5

(51) Int. Cl. A61B 5/00; A61B 5/145; G16H 20/17; G16H 40/63; G16H 50/20; G16H 50/30

(52) U.S. Cl. CPC A61B 5/7292; A61B 5/14503; A61B 5/14532; A61B 5/7282; A61B 5/742; A61B 5/746; G16H 20/17; G16H 40/63; G16H 50/20; G16H 50/30

(57) ABSTRACT

Methods for predicting glucose values which involve determining a predicction time window using historical data indicative of glucose level influencing events of a person having diabetes and at least one predicted glucose level influencing event. Further disclosed are data processing systems for predicting glucose values, medical servers, user devices, and computer programs.

### CROSS REFERENCE TO RELATED APPLICATIONS

This is a continuation of application serial no. PCT/EP2024/058059 filed Mar. 26, 2024 which claims priority from EP 23 167 970.5 filed Apr. 14, 2023 and from US 63/493,336 filed Mar. 31, 2023, the entire disclosures of each of which are hereby incorporated herein by reference.

### BACKGROUND

The present disclosure refers to computer-implemented methods for predicting glucose values. Further, the present disclosure refers to a data processing system for predicting glucose values, a medical server, a user device, and a computer program.

People may suffer from either Type I or Type II diabetes, in which the sugar level in the blood is not properly regulated by the body. Many of these people may use continuous glucose monitoring (CGM) to monitor their glucose level on an ongoing basis. In order to perform CGM, a glucose sensor may be placed under the skin of a person having diabetes for measuring the glucose level in the interstitial fluid. The glucose sensor may periodically measure the glucose level, such as every one minute, and transmit the results of the glucose measurement result to an insulin pump, blood glucose meter, smart phone or other electronic monitor. For predicting glucose values, typically static prediction time windows are employed.

As set out in document US 2022/0061712 A1, data describing glucose measurements are received from a continuous glucose monitoring system worn by a user and predicted glucose values during a future time period are generated for the user based on the data. A determination is made that at least one of the predicted glucose values satisfies a threshold value for an alert, which is associated with a prediction horizon that defines an amount of time prior to satisfaction of the threshold value for communicating the alert to the user.

Document US 2020/098464 A1 discloses a method of monitoring a physiological condition of a patient that involves obtaining data indicative of a current state of the patient, identifying one or more historical patient states similar to the current state of the patient based on historical data associated with the one or more historical patient states maintained in a database, obtaining a model for the physiological condition of the patient in the future from the current state. The model is determined based on the historical data associated with the one or more historical patient states.

In document U.S. Pat. No. 11,229,406 B2, a method of monitoring a physiological condition of a patient is described that involves obtaining current measurement data for the physiological condition of the patient provided by a sensing arrangement, obtaining a user input indicative of future events associated with the patient, and in response to the user input, determining a prediction of the physiological condition of the patient in the future based on the current measurement data and the future events using one or more prediction models associated with the patient.

Document US 2017/0177825 A1 discloses a method of determining a level of hypoglycemic unawareness displayed by a patient that includes maintaining a data structure including one or more glucose concentrations, receiving a glucose concentration, and determining a query based upon the received glucose concentration and the data structure.

### SUMMARY

The present disclosure provides improved technologies for predicting glucose values based on historical data of a person having diabetes with an increased level of confidence.

For solving the problem, computer-implemented methods for predicting glucose values as well as a data processing system, a medical server, a user device, and a computer program product are provided. Further embodiments are disclosed hereinbelow.

According to an aspect, a computer-implemented method for predicting glucose values is provided. The method comprises: receiving continuous glucose monitoring data indicative of a glucose level in a bodily fluid from a continuous glucose monitoring sensor device coupled to a person having diabetes; determining, using historical data indicative of glucose level influencing events of the person having diabetes and based on at least one predicted glucose level influencing event, a prediction time window; determining, based on the continuous glucose monitoring data, a plurality of predicted glucose values for the prediction time window; and displaying, at least partially, the plurality of predicted glucose values.

According to another aspect, a computer-implemented method for predicting glucose values is provided, which is carried out in a medical server with at least one processor. The method comprises: receiving, in the medical server from at least one of a continuous glucose monitoring sensor device, which is coupled to a person having diabetes, and a user device coupled to the continuous glucose monitoring sensor device, continuous glucose monitoring data indicative of a glucose level in a bodily fluid; determining, in the medical server, using historical data indicative of glucose level influencing events of the person having diabetes and based on at least one predicted glucose level influencing event, a prediction time window; determining, in the medical server, based on the continuous glucose monitoring data, a plurality of predicted glucose values for the prediction time window; and transmitting, at least partially, the plurality of predicted glucose values from the medical server to the personal data processing device.

According to another aspect, a computer-implemented method for predicting glucose values is provided, which is carried out in a user device with at least one processor. The method comprises: transmitting, from the user device to a medical server, continuous glucose monitoring data indicative of a glucose level in a bodily fluid; receiving, in the user device from the medical server, a plurality of predicted glucose values for a prediction time window, wherein the prediction time window has been determined using historical data indicative of glucose level influencing events of the person having diabetes and based on at least one predicted glucose level influencing event, wherein the plurality of predicted glucose values has been determined based on the continuous glucose monitoring data; and displaying, by the user device, the plurality of predicted glucose values at least partially. Prior to the transmitting of the continuous glucose monitoring data, the continuous glucose monitoring data may be received by the user device from a continuous glucose monitoring sensor device. Alternatively, the continuous glucose monitoring data may be determined, preferably measured, by the user device, in particular by a continuous glucose monitoring sensor device that is part of the user device.

According to another aspect, a data processing system for predicting glucose values comprising at least one processor is provided. The data processing system is configured to receive continuous glucose monitoring data indicative of a glucose level in a bodily fluid from a continuous glucose monitoring sensor device coupled to a person having diabetes; determine, using historical data indicative of glucose level influencing events of the person having diabetes and based on at least one predicted glucose level influencing event, a prediction time window; determine, based on the continuous glucose monitoring data, a plurality of predicted glucose values for the prediction time window; and display, at least partially, the plurality of predicted glucose values.

According to another aspect, a medical server for predicting glucose values comprising at least one processor is provided. The medical server is configured to receive, from at least one of a continuous glucose monitoring sensor device, which is coupled to a person having diabetes, and a user device coupled to the continuous glucose monitoring sensor device, continuous glucose monitoring data indicative of a glucose level in a bodily fluid; determine, using historical data indicative of glucose level influencing events of the person having diabetes and based on at least one predicted glucose level influencing event, a prediction time window; determine, based on the continuous glucose monitoring data, a plurality of predicted glucose values for the prediction time window; and transmit, at least partially, the plurality of predicted glucose values to the user device.

According to another aspect, a user device comprising at least one processor is provided. The user device is configured to transmit, to a medical server, continuous glucose monitoring data indicative of a glucose level in a bodily fluid; receive, from the medical server, a plurality of predicted glucose values for a prediction time window, wherein the prediction time window (30) has been determined using historical data indicative of glucose level influencing events of the person having diabetes and based on at least one predicted glucose level influencing event, wherein the plurality of predicted glucose values has been determined based on the continuous glucose monitoring data; and display, at least partially, the plurality of predicted glucose values. The user device may be configured to receive the continuous glucose monitoring data from a continuous glucose monitoring sensor device. Alternatively, the user device may be configured to determine the continuous glucose monitoring data, preferably to measure the continuous, In particular, the user device may comprise a continuous glucose monitoring sensor device.

Further, a computer program (product) is provided which comprises instructions which, when the computer program is executed by a medical server and/or a user device, cause the medical server and/or the user device to carry out a method for predicting glucose values. Further (sub-) computer programs may be provided, each of which comprise instructions which, when being executed by the medical server or the user device, cause the medical server or, respectively, the user device to carry out a respective method for predicting glucose values.

By determining the prediction time window ahead of determining the plurality of predicted time window, computational efficiency may be increased. Further, traffic between different devices, e.g., a medical server, a user device and a sensor device, may be reduced. Given that battery power on medical devices and user devices such as mobile phones is limited, the solutions provided according to the disclosure may be associated with the benefit of supporting an extended use time of these devices compared to solutions previously known in the art. By the same token, reduction in power consumption may also be associated with the benefit that, for a given medical device, a smaller battery may be chosen which may allow manufacturers to build smaller devices which are more user friendly to carry around in particular in the case of body worn medical devices such as continuous glucose sensor devices. Furthermore, the present disclosure is inter alia associated with the advantage that the person with diabetes may receive less glucose monitoring information that is irrelevant to manage his disease more safely and more effectively because using the embodiments disclosed herein, the displayed information about the predicted glucose values may be tailored in a way that takes into account learnings from the historical data, e.g., from that person. By way of illustration, it is supposed: if it is predicted that there is a high probability that the person's glucose level will fall below a hypoglycemic threshold in 60 minutes, this would, using hitherto known systems, usually trigger to informing the patient about this predicted hypoglycemia so he can take action to prevent this. Now with the present disclosure, with a prediction time window of 60 minutes, the display time interval for which predicted glucose values are actually displayed may, e.g., be shortened to 30 minutes in case based on the available historical data it is determined that it is very likely that the person will take a meal in the next 30 minutes, which meal consumption would, in turn, result in an increase in the glucose level. This increase, in turn, may likely prevent that the person's glucose level will fall below the hypoglycemic threshold in 60 min. Accordingly, it may be misleading to the person to inform him about the predicted glucose level in a time window of 60 minutes.

In line with the present disclosure, contrary to the hitherto known models with a static prediction time window, the dynamically adjustable predictive time window may thus allow to respond more appropriately in the presence of expected glucose level influencing events that have an impact on the glucose level prediction and what information a person needs to manage his diabetes. As a result, the subject matter presented herein may enable the patient to benefit from a more personalized management of his or her glycemic state and its predicted changes. This way the patient may receive fewer insignificant or nuisance alarms compared to available systems which alleviates the patient's burden to manage his diabetes disease. This in turn may also improve the overall patient compliance in diabetes management over time.

The determining of the prediction time window may comprise determining a size/(time) length of the prediction time window. The at least one predicted glucose level influencing event may be a future event at the time the prediction time window is determined. The plurality of predicted glucose values may be determined free and/or independent from the at least one predicted glucose level influencing event. Alternatively, the determining of the plurality of predicted glucose values may be based on the at least one predicted glucose level influencing event.

The prediction time window may be determined based on a probability of the at least one predicted glucose level influencing event occurring, for example within a predetermined influencing event time window. The predetermined time interval may be longer than, as long as, or shorter than the prediction time window. The method may comprise determining the probability of the at least one predicted glucose level influencing event occurring (preferably within the predetermined influencing event time window), in particular using the historical data. For example, using the historical data, the probability of the person having diabetes having a meal in the next hour may have been determined to be 85%. Generally, the method may comprise predicting the at least one predicted glucose level influencing event, preferably within the predetermined influencing event time window.

The predetermined influencing event time window and or the prediction time window may start from a current time (present time) and/or extend to future times (for which glucose values may be predicted). For example, the predetermined influencing event time window and/or the prediction time window may have a length of time between 30 minutes and 600 minutes, preferably between 45 minutes and 360 minutes, more preferably between 60 minutes and 180 minutes.

The prediction time window may be determined based on the probability (that the at least one predicted glucose level influencing event will occur, preferably within the predetermined time interval) exceeding a predetermined upper probability threshold or falling below a predetermined lower probability threshold.

The upper probability threshold may be, e.g., at least 50%, preferably at least 75%, more preferably at least 90%. The lower probability threshold may be, c.g., at most 20%, preferably at most 10%, more preferably at most 5%.

The determining a prediction time window may further comprise determining whether a predicted glucose level influencing event exists within a predetermined influencing event time window; in case no predicted glucose level influencing event exists within the predetermined influencing event time window, determining the prediction time window (30) to be equal to a predetermined standard time window; and in case at least one predicted glucose level influencing event exists within the predetermined influencing event time window, determining the prediction time window (30) based on at least one predicted glucose level influencing event. Thereby, it may be assured that a sensible prediction time window is determined even if there is no glucose level influencing event predicted for a relevant future time frame as defined by the predetermined influencing event time window. The predetermined standard time window may be a time window that is an adequate time window in which glucose predictions are to be presented to a person with diabetes absent special circumstances. Herein, the predetermined influencing event time window may be equal to the predetermined standard time window, so that the prediction time window is adapted in case relevant events are predicted for a standard time window for displaying glucose predictions to a person with diabetes. Alternatively, the predetermined influencing event time window may differ in (time) length from the predetermined standard time window, in particular to ensure secure prediction of potentially relevant events while ensuring efficiency.

Determining the plurality of predicted glucose values may further be based on at least one of the following: meal event information, insulin bolus information, insulin basal amounts, physical activity event information, stress event information, illness event information. In particular, the predicted glucose values may be determined based on a recent or planned meal consumption (i.e., carbohydrate intake), insulin bolus amounts, insulin basal amounts, physical activity level and duration, stress, and/or illness. Alternatively, or additionally, other indicators and parameters utilized in bolus calculators or automated insulin delivery systems known in the art may be employed in determining the plurality of predicted glucose values.

The method may comprise, based on the plurality of predicted glucose values, displaying one of a shortened display time interval comprising a subset of the plurality of predicted glucose values, an extended display time interval comprising further predicted glucose values (in particular based on determining an extended prediction time window and determining further predicted glucose values for the extended prediction time window), and (each of) the plurality of predicted glucose values.

In particular, the method may comprise, based on the plurality of predicted glucose values, determining a shortened display time interval, which is shorter than the prediction time window, comprising a subset of the plurality of the plurality of predicted glucose values and displaying the subset of the plurality of predicted glucose values. The determining of the shortened time display interval may, for example, be based on a predicted glucose value trend. For example, the determining of the shortened display time interval may be based on an estimated impact of the at least one predicted glucose level influencing event on the plurality of predicted glucose values (without considering the at least one predicted glucose level influencing event). For determining the impact of the at least one predicted glucose level influencing event on the plurality of predicted glucose values, e.g., an adjusted plurality of predicted glucose values may be determined. Alternatively, a metric indicative of a glucose level trend may be determined. The method may comprise displaying the adjusted plurality of predicted glucose values.

The shortened display time interval may be determined in the medical server and/or the user device. The method may comprise transmitting (only) the subset of the plurality of predicted glucose values from the medical server to the user device.

The determining of the shortened display time interval may be based on at least one of the plurality of predicted glucose values exceeding a predetermined upper glucose threshold or falling below a predetermined lower glucose threshold. Further, the determining of the shortened display time interval may be based on the estimated impact of the at least one predicted glucose level influencing event on the plurality of predicted glucose values exceeding the predetermined upper glucose threshold or falling below the predetermined lower glucose threshold. For example, a shortened display time interval may be determined in at least one of the following cases:
predicted meal consumption (and/or prediction of no fasting), drop of the predicted glucose values to hypoglycemic levels, and rising glucose trend after meal consumption,
predicted insulin administration, rise of the predicted glucose values to hyperglycemic levels, and stopping of rise after insulin administration, and
predicted physical exercise, rise of the predicted glucose values to hyperglycemic levels, and stopping of rise due to physical exercise.

In a specific example, a meal consumption may be determined as predicted glucose level influencing event and the plurality of predicted glucose values (without considering the historical data) may indicate a drop to hypoglycemia levels (fall below the hypoglycemia threshold). Yet, as a result of the predicted meal consumption, a hypoglycemia risk may be determined to be overcome. The displayed interval may thus be shortened so that the person having diabetes is not shown predicted glucose values indicative of hypoglycemia. Similarly, if predicted insulin administration is determined to stop a predicted rise of glucose values to hyperglycemic levels, the displayed time interval may be shortened so that the predicted hyperglycemia is not shown, or, if a predicted physical exercise is determined to stop a predicted rise of glucose values to hyperglycemic levels, the displayed time interval may be shortened so that the predicted hyperglycemia is not shown.

The determining of the shortened display time interval may be based on the adjusted plurality of predicted glucose values being below the predetermined upper glucose threshold and/or above the predetermined lower glucose threshold. The upper glucose threshold may for example be indicative of hyperglycemia (e.g., 180 mg/dL or 250 mg/dL). The lower glucose threshold may be indicative of hypoglycemia (e.g., 70 mg/dL or 54 mg/dL).

The shortened display time interval may for example exclude predicted glucose values subsequent to a time distance after the expected time of the at least one predicted glucose level influencing event occurring, in particular subsequent to a time distance after a confidence (time) interval around the expected time. The confidence interval may be a p confidence interval with p being at least 70%, preferably at least 85%, more preferably at least 95%. The shortened time interval may thus exclude, e.g., predicted glucose values impacted by meal consumption.

The shortened display time interval may be determined so as to exclude the expected time of the at least one predicted glucose level influencing event occurring. For example, a (second) time interval that includes the expected time of the at least one of the glucose level influencing events occurring may be excluded from the shortened display time interval.

The shortened display time interval may be determined so as to exclude predicted glucose values falling into a time frame for which the predicted glucose values have been determined to exhibit a variability that is greater than a threshold. In particular, absolute and/or relative error bounds may be provided. Thereby, predicted glucose values for a time in which a prediction is noisy or has a prediction range that is too wide to be of value to a user may be excluded from being displayed.

Additionally, or alternatively, the method may comprise, based on the plurality of predicted glucose values, determining an extended prediction time window, which is longer than the prediction time window, and determining a plurality of further predicted glucose values for the extended prediction time window. Herein, the further predicted glucose values may be displayed in addition to the plurality of predicted glucose values. Alternatively, the further predicted glucose values may not be displayed or may only be displayed in part. In this context, the embodiment described in detail above with reference to the shortened display time interval may apply accordingly. The extended prediction time window and/or the further predicted glucose values may be determined in the medical server and/or the user device. The further predicted glucose values may be determined based on the continuous glucose monitoring data. The further predicted glucose values may temporally follow the plurality of predicted glucose values. The method may comprise transmitting the further predicted glucose values from the medical server to the user device.

The determining of the extended prediction time window and/or which of the further predicted glucose values are displayed may be based on the predicted glucose value trend. For example, the determining of the extended prediction time window and/or which of the further predicted glucose values are displayed may be based on an estimated impact of the at least one predicted glucose level influencing event on the plurality of predicted glucose values (without considering the at least one predicted glucose level influencing event).

The determining of the extended prediction time window and/or which of the further predicted glucose values are displayed may be based on at least one of the plurality of predicted glucose values exceeding a predetermined upper glucose threshold or falling below a predetermined lower glucose threshold. Further, the determining of the extended prediction time window and/or which of the further predicted glucose values are displayed may be based on the estimated impact of the at least one predicted glucose level influencing event on the plurality of predicted glucose values exceeding the predetermined upper glucose threshold or falling below the predetermined lower glucose threshold. The determining of the extended prediction time window and/or which of the further predicted glucose values are displayed may be based on the adjusted plurality of predicted glucose values being above the predetermined upper glucose threshold and/or below the predetermined lower glucose threshold.

For example, an extended prediction time window may be determined in at least one of the following cases:
predicted fasting, in which case the predicted glucose values and further predicted glucose values displayed may be based on a predicted drop of the predicted glucose values to hypoglycemic levels,
predicted fasting, in which case the predicted glucose values and further predicted glucose values displayed may be based on a predicted rise of the predicted glucose values to hyperglycemic levels,
predicted sleeping, in which case the predicted glucose values and further predicted glucose values displayed may be based on a predicted drop of the predicted glucose values to hypoglycemic levels, and
predicted sleeping, in which case the predicted glucose values and further predicted glucose values displayed may be based on a predicted rise of the predicted glucose values to hyperglycemic levels.

In a specific example, sleep may be determined as predicted glucose level influencing event. Since the person having diabetes is expected to sleep, the prediction time window may be extended so that a longer prediction is performed. In this case, the further predicted glucose values may be displayed entirely or in part so the person is shown (further) predicted glucose values indicative of hypoglycemia, or the person may not be shown the further predicted glucose values in line with the embodiments laid out above.

The determining of the extended prediction time window may comprise extending the prediction time window until the probability of the at least one predicted glucose level influencing event occurring or being completed exceeds a minimum probability threshold. The minimum probability threshold may, e.g., be between 5% and 50%. For example, the probability of meal consumption may be reduced at night and the prediction time window may be extended accordingly.

The determining of the extended time interval may also comprise extending the prediction time window by a fixed factor, e.g., between 1.1 and 10, preferably between 1.5 and 3.

Additionally, or alternatively, the method may comprise, based on the plurality of predicted glucose values, determining that (each of) the plurality of predicted glucose values are to be displayed. This may preferably be the case when neither a shortened display time interval nor an extended prediction time window are determined. The displaying of (each of) the plurality of predicted glucose values (and/or the determining thereof) may be based on a predicted glucose value trend. The determining that (each of) the plurality of predicted glucose values are to be displayed may be based on the estimated impact of the at least one predicted glucose level influencing event on the plurality of predicted glucose values (without considering the glucose level influencing event).

For example, (each of) the plurality of predicted glucose values may be determined to be displayed in at least one of the following cases:
predicted meal consumption (and/or prediction of no fasting), rise of the predicted glucose values to hyperglycemic levels, and further rising glucose trend after meal consumption,
prediction of no meal consumption and drop of the predicted glucose values to hypoglycemic levels,
prediction of no meal consumption and rise of the predicted glucose values to hyperglycemic levels,
predicted insulin administration, drop of the predicted glucose values to hypoglycemic levels, and further drop after insulin administration,
predicted physical exercise, drop of the predicted glucose values to hypoglycemic levels, and further drop due to physical exercise,
prediction of no sleep, drop of the predicted glucose values to hypoglycemic levels, and unchanged glucose trend due to absence of sleep, and
prediction of no sleep, rise of the predicted glucose values to hyperglycemic levels, and unchanged glucose trend due to absence of sleep.

The method may comprise determining an alert or alarm event, e.g., in case of hypoglycemia or hyperglycemia. In particular, determining the alarm event may be based on the at least one predicted glucose level influencing event and/or on the plurality of predicted glucose values, in particular, on at least one of the plurality of predicted glucose values exceeding a predetermined upper glucose threshold or falling below a predetermined lower glucose threshold. Based on (and/or in case of) the alarm event, an alarm may be output. Outputting the alarm, in particular an alarm intensity, may further depend on a hypoglycemia awareness and/or hyperglycemia awareness of the person having diabetes. The alarm intensity may, e.g., correspond to a sound and/or light level. The hypoglycemia awareness and/or hyperglycemia awareness may, e.g., be determined from the historical data and/or be provided via user input. For example, a user may be alerted before going to sleep while they are still awake to raise awareness of a predicted hypoglycemic episode later. In another example, a user may be alerted when in a sleep state. In this case, alarm intensity may be modified, for example raised to wake the user. In a further example, alarm intensity may additionally be modified based on hypoglycemic awareness.

The method may comprise suppressing an alarm output, in particular for hypoglycemia. Preferably, suppressing the alarm output may be overridden in case the glucose level falls below a critical threshold.

The alarm (output) may be provided via an output device and/or an alarm device, e.g., a speaker and/or a display.

The plurality of predicted glucose values may be displayed via the output device. Displaying the plurality of predicted glucose values may comprise excluding a display of predicted glucose values outside the prediction time window and/or outside the shortened display time interval. The same may apply, accordingly to displaying the plurality of further predicted glucose values.

The method may comprise determining the at least one predicted glucose level influencing event, preferably using the historical data, in particular using a statistical model based on the historical data. In this case, it may be confirmed that the historical data fulfills one or more predetermined data sufficiency criteria before determining the at least one predicted glucose level influencing event. For example, the sufficiency criteria may relate to sleep or fasting which involved the “absence” of recording meal information. Determining the at least one predicted glucose level influencing event may, e.g., be carried out in the medical server and/or the user device.

In particular, the probability of the at least one predicted glucose level influencing event occurring may be determined using a statistical model based on the historical data. In general, determining the at least one predicted glucose level influencing event and/or the prediction time window may be carried out using a statistical model based on the historical data.

The statistical model may be trained using the historical data as training data, e.g., using a machine learning algorithm. The historical data may comprise times (of day) and/or dates of the glucose level influencing events (for example, meal consumption time stamps). The historical data may further comprise continuous glucose monitoring data, in particular, glucose values for the respective times before and subsequent to the glucose level influencing events. The statistical model may also be determined using linear regression. The probability of the at least one predicted glucose level influencing event occurring also be determined using a histogram based on preceding glucose level influencing events.

The probability of the at least one predicted glucose level influencing event occurring may be determined using correlation information (in particular time correlation information), e.g., between different types of glucose level influencing events and/or between different glucose level influencing events of the same type. For example, physical exercise may be correlated with meal consumption one hour later. Further, meal consumption may decrease the probability of further meal consumption within the next hours.

The predicted glucose values may be determined using one or a plurality of prediction algorithms, preferably from the continuous glucose monitoring data. In particular, the predicted glucose values may be determined using a plurality of prediction algorithms by determining a (weighted or unweighted) average of prediction values of each of the plurality of prediction algorithms. Specifically, a hyperglycemia event and/or a hypoglycemia event may be determined by determining the (weighted or unweighted) average of prediction values of each of the plurality of prediction algorithms. The one or the plurality of prediction algorithms may be trained using the historical data as training data. The predicted glucose values may be determined using a physiological model for glucose level prediction.

Determining the prediction time window and/or determining the extended prediction time window and/or the displaying of the shortened display time interval may additionally be based on a time of day. In particular, the probability that the at least one predicted glucose level influencing events will occur may be determined additionally based on the time of day. For example, the probability of meal consumption or physical exercise may be lower at night and may be correspondingly determined from the historical data.

Determining the prediction time window and/or determining the extended prediction time window and/or the displaying of the shortened display time interval may additionally be based on a frequency of the at least one of the glucose level influencing events (e.g., per day). For example, in case the person having diabetes were to cat only one or two meals per day, the prediction time window may be expanded accordingly.

The glucose level influencing events may comprise (or consist of) glucose level influencing actions (in particular, intervening actions), for example performed by the person having diabetes.

The historical data may consist of or include patient-based (individual) historical data. For example, the historical data may have been determined using patient-specific data (only). Additionally, or alternatively, the historical data may include population-based historical data and/or subpopulation-based historical data. Subpopulation-based historical data may for example include gender-specific data (e.g., male/female) and/or diabetes type-specific data (e.g., diabetes type I/II).

The glucose level influencing events may comprise at least one of (or consist of) meal consumption (i.e., carbohydrate intake), insulin bolus administration, physical exercise, fasting, and sleep. Each of the glucose level influencing events may generally comprise at least one of meal consumption, insulin bolus administration, increased physical activity, and decreased physical activity.

The method may further comprise receiving user data indicative of a further glucose level influencing event having occurred during and/or subsequent to the prediction time window and/or the extended prediction time window and, preferably, modifying the historical data based on the user data. Thus, the method may further comprise updating the statistical model and/or the prediction algorithm(s) based on the modified historical data.

The continuous glucose monitoring data may comprise glucose values. A glucose level and/or glucose values may be determined by continuous glucose monitoring via a fully or partially implanted sensor and/or worn sensor. In general, in the context of continuous glucose monitoring, a glucose value or glucose level in a bodily fluid may be determined. The glucose level or value may be, e.g., subcutaneously measured in an interstitial fluid. Continuous glucose monitoring may be implemented as a nearly real-time or quasi-continuous monitoring procedure frequently or automatically providing/updating analyte values without user interaction.

The embodiments described above in connection with the method for predicting glucose values may be provided correspondingly for each of the further methods according to the disclosure, the data processing system for predicting glucose values, the medical server for predicting glucose values and/or the user device. Within the context of the present disclosure, the indication of an interval using the terms “between . . . and” includes the limit points of the interval.

### BRIEF DESCRIPTION OF THE DRAWINGS

The above mentioned and other features of the various embodiments, and the manner of attaining them, will become more apparent and will be better understood by reference to the following description of exemplary embodiments taken in conjunction with the accompanying drawings, wherein:

FIG. 1 shows a graphical representation of a system for predicting glucose values.

FIG. 2 shows a graphical representation of a method for predicting glucose values.

FIG. 3a shows a graphical representation of a glucose curve with a prediction time window.

FIG. 3b shows a graphical representation of a glucose curve with another prediction time window.

Corresponding reference characters indicate corresponding parts throughout the several views. Although the exemplification set out herein illustrates embodiments of the invention, in several forms, the embodiments disclosed herein are not intended to be exhaustive or to be construed as limiting the scope of the invention to the precise forms disclosed.

### DETAILED DESCRIPTION

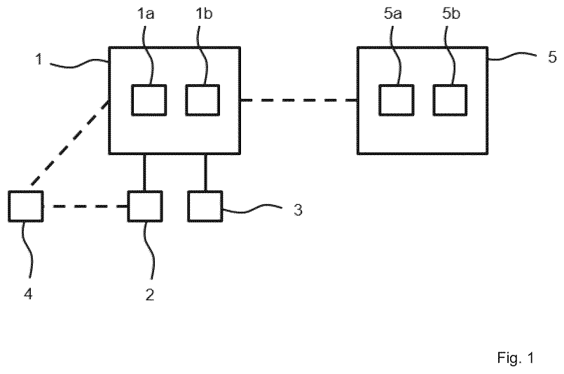

FIG. 1 shows a graphical representation of a (data processing) system for predicting glucose values. The system comprises a user device 1 (personal data processing device) provided with one or more processors 1a and a memory 1b for storing machine readable instructions. The user device 1 is connected to an input device 2 configured to receive (user) input data and an output device 3 configured for outputting data. The input device 2 and the output device 3 may or may not be implemented integrally with the user device 1.

The one or more processors 1a may be a controller, an integrated circuit, a microchip, a computer, or any other computing device capable of executing machine readable instructions. The memory 1b may be RAM, ROM, a flash memory, a hard drive, or any device capable of storing machine readable instructions.

The one or more processors 1a may be integral with a single component of the system. The one or more processors 1a may also be separately located within discrete components such as, for example, a glucose meter, a medication delivery device, a mobile phone, a portable digital assistant (PDA), a mobile computing device such as a laptop, a tablet, or a smart phone.

The output device 3 may be configured to provide graphical, textual and/or auditory information. The output device 3 may include an electronic display such as, for example, a liquid crystal display, a thin film transistor display, a light emitting diode display, a touch screen, or any other device capable of transforming signals from a processor into an optical output, or a mechanical output, such as, for example, a speaker or a printer for displaying information.

The user device 1 and/or the input device 2 may comprise or be coupled to a continuous glucose monitoring sensor device 4 for providing biological data indicative of properties of an analyte and/or a continuous glucose monitoring system coupled to a person having diabetes. The input device 2 may be configured to receive raw data from the continuous glucose monitoring sensor device 4 and process the raw data into glucose monitoring data and, preferably, transmit the glucose monitoring data to the user device 1.

The continuous glucose monitoring sensor device 4 may be configured to detect glucose levels (e.g., glucose concentrations) when coupled to (in particular, worn by) a person having diabetes. For example, the continuous glucose monitoring sensor device 4 can be a disposable glucose sensor that is, e.g., worn under the skin.

The user device 1 may for example be a remote control for the continuous glucose monitoring sensor device 4.

The system further comprises a medical server 5 communicatively coupled with and/or connected to the user device 1. The medical server 5 may comprise one or more processors 5a and a memory 5b.

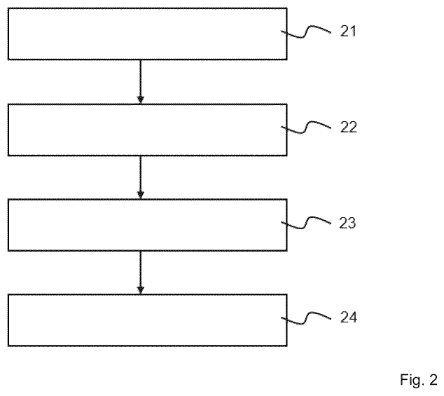

FIG. 2 shows a graphical representation of a computer-implemented method for predicting glucose values.

In a first step 21, continuous glucose monitoring data indicative of a glucose level in a bodily fluid are received in the user device 1 and/or the medical server 5 from the continuous glucose monitoring sensor device 4 coupled to the person having diabetes. The continuous glucose monitoring data may also be relayed from the continuous glucose monitoring sensor device 4 via the personal user device 1 to the medical server 5.

In a second step 22, a prediction time window 30 is determined using historical data indicative of glucose level influencing events of the person having diabetes and based on at least one predicted glucose level influencing event. The (predicted) glucose level influencing events include, e.g., meal consumption, insulin bolus administration, physical exercise, fasting, and/or sleep. The prediction time window 30 may in particular be determined based on a probability of the at least one predicted glucose level influencing event occurring within a predetermined time interval. For example, the probability of the person having diabetes having a meal in the next hour may be 85% and the prediction time window 30 may be determined correspondingly. In case no glucose level influencing event is predicted, the prediction time window is set to a default value (predetermined standard time window).

The prediction time window 30 may be determined in the medical server 5 and/or the user device 1. The historical data may be stored in the memory 5b of the medical server 5 and/or the memory 1b of the user device 1. The historical data may be received and/or created and/or modified by the medical server 5 and/or the user device 1, e.g., via the input device 2.

In a third step 23, a plurality of predicted glucose values 32 are determined for the prediction time window 30 based on the continuous glucose monitoring data.

The plurality of predicted glucose values 32 may be determined in the medical server 5 and/or in the user device 1.

In a fourth step 24, the plurality of predicted glucose values 32 are (at least partially) displayed to the person having diabetes, e.g., via the user device 1, in particular, the output device 3.

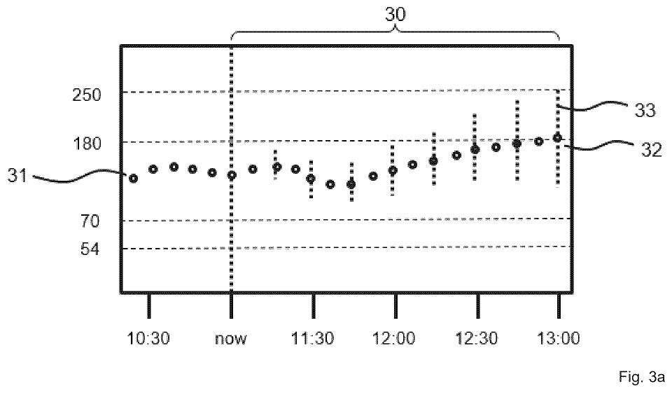

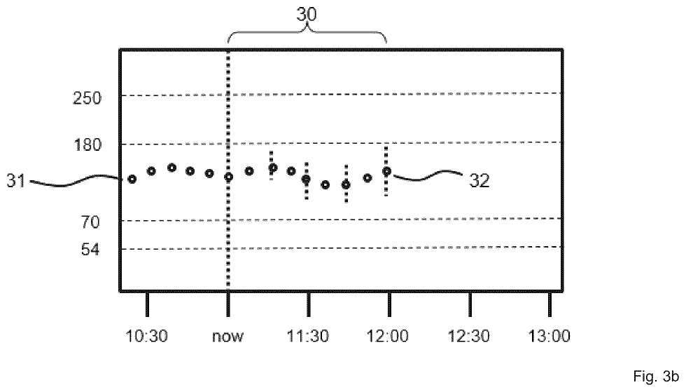

FIGS. 3a and 3b show graphical representations of glucose curves with prediction time windows 30 of different length. The glucose curves comprise measured glucose values 31 (as part of the continuous glucose monitoring data) and predicted glucose values 32 for the prediction time window 30. Confidence intervals for the predicted glucose values 32 are represented by bars 33.

Depending on the historical data and the predicted glucose level influencing event, the prediction window may be longer (FIG. 3a) or shorter (FIG. 3b). In addition, based on the plurality of predicted glucose values 32, only a shortened display time interval comprising a subset of the plurality of predicted glucose values 32 may be displayed or an extended prediction time window may be determined for which further predicted glucose values are determined which may then be displayed.

Prediction of a hypoglycemia event or a hyperglycemia event one or more hours in the future may not be relevant in case the person having diabetes is about to eat within the prediction time window 30. If, for example, a hypoglycemia event is predicted to occur within the next hour, there is also a high probability of meal consumption within the next 30 minutes and the prediction time window 30 may be correspondingly shortened with respect to the predetermined time interval or the prediction time window 30 may not be shortened when it is determined based on the predicted hypoglycemia event but predicted glucose values may only be displayed for a shortened display time interval because the meal consumption event makes it likely that the predicted hypoglycemia event will not occur.

In another more specific example, the person having diabetes may typically cat a meal at noon. This habit may be represented by historical data which provides, e.g., a probability of 90% for meal consumption between 12:00 and 12:15. Still, the meal consumption, which is about to take place, may not have been entered by the person having diabetes. Without shortening the time interval for the displayed predicted glucose values and not yet taking into account the latest meal consumption, a hypoglycemia event would have been displayed as predicted for 13:00.

The prediction time window 30 and/or the display time interval may be even shorter for persons with diabetes who consume (and document) their meal consumption within a narrow time window every day.

In another example, the person having diabetes may, according to the historical data, have a high probability of performing exercise when the glucose level is elevated for an extended period (for example, after a meal). The display time interval may correspondingly be shorter.

If, on the other hand, the person having diabetes is, based on historical data, unlikely to intervene to influence their glucose level in a larger time frame (e.g., by meal consumption or bolus administration), the prediction time window 30 may be expanded. In this case, further predicted glucose values may be displayed or only the (original) predicted glucose values may be displayed or even only predicted glucose value for a shortened display time interval may be displayed in the case of predicted glucose values becoming hypo-or hyperglycemic. A low probability of a glucose level influencing action may appear, e.g., during bedtime, overnight time, or fasting. The probability that at least one of the glucose level influencing events will occur may thus be determined based on the time of day.

The person having diabetes may record which glucose level influencing event has when taken place in reaction to the display of the plurality of predicted (and optionally further predicted) glucose values 32. The historical data may be updated accordingly, resulting in a more appropriate glucose level prediction with respect to glucose level influencing events.

In the following table 1, exemplary embodiments in connection with the impact of predicted glucose level influencing events and predicted glucose value trends. For each category of predicted glucose level influencing events (col. 1), specific events predicted from the historical data are listed in col. 2 and combined with different predicted glucose value trends (col. 3). The respective impact of cach event on the glucose value trend and on the displayed interval of predicted glucose values 32 is shown in cols. 4 and 5.

TABLE 1
Impact of predicted glucose level influencing events and predicted glucose value trends

| | | Predicted | | |
|---|---|---|---|---|
| | | glucose value | | Impact on |
| | | trend | | displayed |
| | | (prediction | | interval of |
| Category of | | time | Impact of | predicted |
| predicted | Specific event | window) | event on | glucose values |
| glucose level | predicted from | without | glucose | with respect |
| influencing | historical | considering | value | to prediction |
| events | data | historical data | trend | time window |
| Meal | Meal | Drop to | Rise after meal | Displayed interval is |
| consumption | consumption | hypoglycemia | | shorter since expected |
| | expected in near | | | meal consumption will |
| | future | | | overcome hypoglycemia |
| | | | | risk |
| | Meal | Rise to | Rise after meal | No change in displayed |
| | consumption | hyperglycemia | (quicker rise to | interval (maintain |
| | expected in near | | hyperglycemia) | prediction time window |
| | future | | | for display) since |
| | | | | intervention would not |
| | | | | result in avoiding |
| | | | | hyperglycemia threshold |
| | No meal | Drop to | Drop to | Maintain prediction time |
| | consumption | hypoglycemia | hypoglycemia | window for display to |
| | expected | | | visualize hypoglycemia |
| | | | | risk |
| | No meal | Rise to | Rise to | Maintain prediction time |
| | consumption | hyperglycemia | hyperglycemia | window for display to |
| | expected | | | visualize hyperglycemia |
| | | | | risk |
| Insulin | Insulin | Drop to | Quicker drop | Maintain prediction time |
| administration | administration in | hypoglycemia | to | window for display since |
| | near future | | hypoglycemia | intervention would not |
| | | | | result in avoiding |
| | | | | hypoglycemia threshold |
| | Insulin | Rise to | Stop rise to | Display interval shorter |
| | administration in | hyperglycemia | hyperglycemia | than prediction time |
| | near future | | or drop in | window if hyperglycemia |
| | | | glucose level | is stopped/ compensated |
| Physical | Physical exercise | Drop to | Quicker drop | Maintain prediction time |
| exercise | planned in near | hypoglycemia | to | window for display since |
| | future | | hypoglycemia | intervention would not |
| | | | | result in avoiding |
| | | | | hypoglycemia threshold |
| | Physical exercise | Rise to | Stop rise to | Display interval shorter |
| | planned in near | hyperglycemia | hyperglycemia | than prediction time |
| | future | | or drop in | window if hyperglycemia |
| | | | glucose level | is stopped/compensated |
| Fasting | Fasting expected | Drop to | Drop to | Extend to longer |
| | in near future (no | hypoglycemia | hypoglycemia | prediction time window |
| | meal | | | to evaluate hypoglycemia |
| | consumption in | | | risk |
| | near future) | | | |
| | Fasting expected | Rise to | Rise to | Extend to longer |
| | in near future (no | hyperglycemia | hyperglycemia | prediction time window |
| | meal | | | to evaluate hyperglycemia |
| | consumption in | | | risk |
| | near future) | | | |
| | No fasting | Drop to | Rise after meal | Display interval shorter |
| | expected in near | hypoglycemia | | than prediction time |
| | future (meal | | | window since meal |
| | consumption in | | | consumption will |
| | near future) | | | overcome |
| | | | | hypoglycemia risk |
| | No fasting | Rise to | Rise after meal | Maintain prediction time |
| | expected in near | hyperglycemia | (quicker rise to | window for display since |
| | future (meal | | hyperglycemia) | intervention would not |
| | consumption in | | | result in avoiding |
| | near future) | | | hyperglycemia threshold |
| Sleep | Sleep expected in | Drop to | Drop to | Extend to longer |
| | near future (no | hypoglycemia | hypoglycemia | prediction time window |
| | meal | | | to evaluate hypoglycemia |
| | consumption in | | | risk and output alarm |
| | near future and | | | (while user still expected |
| | goal to avoid | | | to be awake) |
| | alarms during | | | |
| | sleep → find | | | |
| | solution before | | | |
| | sleep) | | | |
| | Sleep expected in | Rise to | Rise to | Extend to longer |
| | near future (no | hyperglycemia | hyperglycemia | prediction time window |
| | meal | | | to evaluate hyperglycemia |
| | consumption in | | | risk and output alarm |
| | near future and | | | (while user is still |
| | goal to avoid | | | expected to be awake) |
| | alarms during | | | |
| | sleep → find | | | |
| | solution before | | | |
| | sleep)) | | | |
| | No Sleep | Drop to | Situation | Maintain prediction time |
| | expected in near | hypoglycemia | unchanged | window |
| | future | | | |
| | No Sleep | Rise to | Situation | Maintain prediction time |
| | expected in near | hyperglycemia | unchanged | window |
| | future | | | |

The features disclosed in this specification, the figures and/or the claims may be material for the realization of various embodiments, taken in isolation or in various combinations thereof.

While this invention has been described as having exemplary designs, the present invention may be further modified within the spirit and scope of this disclosure. This application is therefore intended to cover any variations, uses, or adaptations of the invention using its general principles.

What is claimed is:

1. A method for predicting glucose values, comprising:
receiving continuous glucose monitoring data indicative of a glucose level in a bodily fluid from a continuous glucose monitoring sensor device coupled to a person having diabetes;
determining, using historical data indicative of glucose level influencing events of the person having diabetes and, based on at least one predicted glucose level influencing event, a prediction time window;
determining, based on the continuous glucose monitoring data, a plurality of predicted glucose values for the prediction time window; and
displaying, at least partially, the plurality of predicted glucose values.

2. The method of claim 1, wherein the prediction time window is determined based on a probability of the at least one predicted glucose level influencing event occurring.

3. The method of claim 2, wherein the prediction time window is determined based on the probability of the at least one predicted glucose level influencing event occurring exceeding a predetermined upper probability threshold or falling below a predetermined lower probability threshold.

4. The method of claim 1, wherein determining a prediction time window further comprises:
determining whether a predicted glucose level influencing event exists within a predetermined influencing event time window;
in case no predicted glucose level influencing event exists within the predetermined influencing event time window, determining the prediction time window to be equal to a predetermined standard time window; and
in case at least one predicted glucose level influencing event exists within the predetermined influencing event time window, determining the prediction time window based on at least one predicted glucose level influencing event.

5. The method of claim 1, wherein determining the plurality of predicted glucose values is further based on at least one of the following: meal event information, insulin bolus information, insulin basal amounts, physical activity event information, stress event information, illness event information.

6. The method of claim 1, further comprising, based on the plurality of predicted glucose values, determining a shortened display time interval, which is shorter than the prediction time window, comprising a subset of the plurality of predicted glucose values and displaying the subset of the predicted glucose values.

7. The method of claim 6, wherein the determining of the shortened display time interval is based on at least one of the plurality of predicted glucose values being above a predetermined upper glucose threshold and/or below a predetermined lower glucose threshold.

8. The method of claim 1, further comprising, based on the plurality of predicted glucose values, determining an extended prediction time window, which is longer than the prediction time window, and determining a plurality of further predicted glucose values for the extended prediction time window.

9. The method of claim 1, further comprising determining an alarm event based on the at least one predicted glucose level influencing event and the plurality of predicted glucose values and outputting an alarm based on the alarm event.

10. The method of claim 1, wherein the determining of the prediction time window is additionally based on a time of day.

11. The method of claim 1, wherein the glucose level influencing events comprise glucose level influencing actions performed by the person having diabetes.

12. The method of claim 1, wherein the glucose level influencing events comprise at least one of meal consumption, insulin bolus administration, physical exercise, fasting, and sleeping.

13. A method for predicting glucose values, the method being carried out in a medical server with at least one processor, the method comprising:
receiving, in the medical server from at least one of a continuous glucose monitoring sensor device coupled to a person having diabetes and a user device coupled to the continuous glucose monitoring sensor device, continuous glucose monitoring data indicative of a glucose level in a bodily fluid;
determining, in the medical server, using historical data indicative of glucose level influencing events of the person having diabetes and based on at least one predicted glucose level influencing event, a prediction time window;
determining, in the medical server, based on the continuous glucose monitoring data, a plurality of predicted glucose values for the prediction time window; and
transmitting, at least partially, the plurality of predicted glucose values from the medical server to the user device.

14. A method for predicting glucose values, the method being carried out in a user device with at least one processor, the method comprising:
transmitting, from the user device to a medical server, continuous glucose monitoring data indicative of a glucose level in a bodily fluid;
receiving, in the user device from the medical server, a plurality of predicted glucose values for a prediction time window, wherein the prediction time window has been determined using historical data indicative of glucose level influencing events of the person having diabetes and based on at least one predicted glucose level influencing event, wherein the plurality of predicted glucose values has been determined based on the continuous glucose monitoring data; and
displaying, by the user device, the plurality of predicted glucose values at least partially.

15. A data processing system for predicting glucose values, the system comprising at least one processor and being configured to:
receive continuous glucose monitoring data indicative of a glucose level in a bodily fluid from a continuous glucose monitoring sensor device coupled to a person having diabetes;
determine, using historical data indicative of glucose level influencing events of the person having diabetes and based on at least one predicted glucose level influencing event, a prediction time window;
determine, based on the continuous glucose monitoring data, a plurality of predicted glucose values for the prediction time window; and
display, at least partially, the plurality of predicted glucose values.

16. A medical server for predicting glucose values, comprising at least one processor configured to:
receive, from at least one of a continuous glucose monitoring sensor device coupled to a person having diabetes and a user device coupled to the continuous glucose monitoring sensor device, continuous glucose monitoring data indicative of a glucose level in a bodily fluid;
determine, using historical data indicative of glucose level influencing events of the person having diabetes and based on at least one predicted glucose level influencing event, a prediction time window;
determine, based on the continuous glucose monitoring data, a plurality of predicted glucose values for the prediction time window; and
transmit, at least partially, the plurality of predicted glucose values to the user device.

17. A user device, comprising at least one processor configured to:
transmit, to a medical server, continuous glucose monitoring data indicative of a glucose level in a bodily fluid;
receive, from the medical server, a plurality of predicted glucose values for a prediction time window, wherein the prediction time window has been determined using historical data indicative of glucose level influencing events of the person having diabetes and based on at least one predicted glucose level influencing event, wherein the plurality of predicted glucose values has been determined based on the continuous glucose monitoring data; and
display, at least partially, the plurality of predicted glucose values.

18. A non-transient computer-readable storage medium, comprising instructions which, when executed by a medical server and/or a user device cause the medical server and/or the user device to carry out the method of claim 1.
